(19) United States
(12) Patent Application Publication
(10) Pub. No.: US 2025/0161028 A1
(43) Pub. Date: May 22, 2025

(54) PATTERNED IMPLANTABLE DEVICES FOR ADDRESSING TUBULAR TISSUE PASSAGEWAY DEFECTS AND METHODS OF MAKING AND USING SAME

(71) Applicants: Georgia Tech Research Corporation, Atlanta, GA (US); EMORY UNIVERSITY, Atlanta, GA (US)

(72) Inventors: Jeong Hun Park, Atlanta, GA (US); Scott J. Hollister, Atlanta, GA (US); Sarah Tucker, Atlanta, GA (US); Andrew Thomas Tkaczuk, Atlanta, GA (US); Mark W. El-Deiry, Atlanta, GA (US)

(21) Appl. No.: 18/841,844

(22) PCT Filed: Mar. 8, 2023
(86) PCT No.: PCT/US2023/063911

§ 371 Date: Aug. 27, 2024

Related U.S. Application Data
(60) Provisional application No. 63/269,006, filed on Mar. 8, 2022.

(51) Int. Cl. A61F 2/20

(52) U.S. Cl. CPC A61F 2/20; A61F 2220/0008

(57) ABSTRACT

An exemplary embodiment of the present disclosure An implantable device. comprising and external wall and a plurality of apertures. The external wall can have an open tubular shape. The plurality of apertures can be located in the external wall. At least a first portion of the plurality of apertures can be configured as suture holes for attaching the implantable device to the user. The implantable device can be configured to be implanted in a passageway of a user.

### CROSS-REFERENCE TO RELATED APPLICATIONS

This application claims the benefit of U.S. Provisional Application Ser. No. 63/269,006, filed on 8 Mar. 2022, which is incorporated herein by reference in its entirety as if fully set forth below.

### GOVERNMENT LICENSE RIGHTS

This invention was made with government support under Agreement No. GR10003058, awarded by National Institutes of Health. The government has certain rights in the invention.

### FIELD OF THE DISCLOSURE

The various embodiments of the present disclosure relate generally to implantable devices, and more particularly to patterned implantable devices for addressing tubular tissue passageway defects in human patients.

### BACKGROUND

Long segmental tracheal defects present a significant reconstructive challenge. In adults, lesions encompassing less than 50% of the tracheal length can be reconstructed with end-to-end anastomosis while defects less than 40% of the native tracheal length in children can be reconstructed. Furthermore, previous interventions that reduce tracheal blood supply including multiple operations, scarring and radiation may reduce these lengths. Attempting end-to-end anastomosis for longer segments or in fields that have been previously operated within may cause significant tension at the anastomosis site that leads to local necrosis, leakage, and potentially stricture and stenosis.

Despite advances in tissue engineering, current reconstructive options for long segment tracheal defects are still extremely limited in the clinic. Allograft implantation of processed cadaveric trachea in pediatric patients requires stenting and has limited success. Deceased-donor non-processed allograft implantation has not been attempted. Clinical trials of a decellularized human donor trachea and the recipient's autologous cells has been made; however, the post-complications were severe with restenosis and collapse of the trachea. Meanwhile, vascularized autogenous tissue has shown significant reconstructive potential over several studies. These vascularized reconstructions, however, require a mechanical support to prevent collapse of the tissue flap and both synthetic and autologous materials were employed. The synthetic materials have been limited to short distance reconstruction and the autologous materials require a separate harvest site with associated risks and potential morbidity to the patient.

Accordingly, there is a need for improved implantable devices that overcome one or more of the disadvantages discussed above.

### BRIEF SUMMARY

An exemplary embodiment of the present disclosure provides an implantable device for implantation in a passageway of a user. The device can comprise an external wall and a plurality of apertures. The external wall can define an interior lumen. The external wall can have an open tubular shape. The plurality of apertures can be disposed in the external wall.

In any of the embodiments disclosed herein, at least a portion of the plurality of apertures can be configured as suture holes for securing the implantable device in the passageway of the patient.

In any of the embodiments disclosed herein, at least a portion of the plurality of apertures can be stent patterned.

In any of the embodiments disclosed herein, the stent patterned apertures can be configured to increase a longitudinal bending flexibility of the implantable device.

In any of the embodiments disclosed herein, the stent patterned apertures can be configured to decrease a radial stiffness of the implantable device.

In any of the embodiments disclosed herein, the suture holes can be arranged in rows on the external wall, and the stent patterned apertures can be positioned between rows of the suture holes.

In any of the embodiments disclosed herein, at least a portion of the plurality of apertures can have an auxetic pattern.

In any of the embodiments disclosed herein, the auxetic patterned apertures can be configured to provide the implantable device with longitudinal and radial expandability.

In any of the embodiments disclosed herein, the implantable device can further comprise an expandable open ring coupled to the external wall. The expandable open ring can be configured to expand radially from a contracted position to an expanded position.

In any of the embodiments disclosed herein, the expandable open ring can be formed from a nonbiodegradable polymer.

In any of the embodiments disclosed herein, the external wall of the implantable device can be formed from a biodegradable polymer.

In any of the embodiments disclosed herein, the expandable open ring can be integral with the external wall.

In any of the embodiments disclosed herein, the expandable open ring can comprise one or more apertures arranged in a zero-Poisson's ratio pattern.

In any of the embodiments disclosed herein, the implantable device can comprise a central core disposed in an interior volume/lumen defined by the external wall.

In any of the embodiments disclosed herein, the implantable device can comprise one or more struts extending outwardly from the central core and coupled to an inner surface of the external wall.

In any of the embodiments disclosed herein, the central core can be detachably coupled to the external wall.

In any of the embodiments disclosed herein, the central core can comprise a reservoir configured to hold a biological substance.

In any of the embodiments disclosed herein, the central core can comprise one or more apertures configured to allow the biological substance to exit the reservoir.

In any of the embodiments disclosed herein, the implantable device can be 3D printed.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-B provide exemplary implantable devices, in accordance with exemplary embodiments of the present disclosure.

FIGS. 2A-C provide exemplary implantable devices, in accordance with exemplary embodiments of the present disclosure.

FIG. 3 provides an exemplary implantable device having a central core, in accordance with an exemplary embodiment of the present disclosure.

FIGS. 4A-D provide an exemplary implantable device with an expandable open ring, in accordance with an exemplary embodiment of the present disclosure.

FIGS. 5A-C provide an exemplary implantable device with an expandable open ring, in accordance with an exemplary embodiment of the present disclosure.

FIGS. 6A-E provide schematics of 3D bioprinting based tubular tissue flap strategy for long segment tracheal reconstruction. FIG. 6A illustrates implantation of the airway scaffold including the core insert and PEGDA hydrogel containing EPO into the latissimus dorsi muscle of a Yucatan minipig. FIG. 6B illustrates retrieval of cylindrical tissue flap composed of the airway scaffold and regenerated tissues. FIG. 6C illustrates incision of both ends of the regenerated tissues surrounding the airway scaffold. FIG. 6D illustrates removal of the core insert from the tissue flap. FIG. 6E illustrates implantation of the tubular tissue flap into the segment tracheal defect.

FIGS. 7A-C illustrates 3D printing and mechanical test results of an exemplary airway scaffold with two stent-patterns. FIG. 7A provides photographs of the printed airway scaffold with two stent-patterns. The load and displacement curves of airway scaffolds under parallel compression (FIG. 7B) and perpendicular compression (FIG. 7C) to the airway scaffold opening.

FIGS. 8A-C illustrate a tubular tissue flap creation after 6 weeks of implantation. FIG. 8A illustrates incision of the muscle tissue flap surrounding the airway scaffold. White arrows indicate blood vessels. FIG. 8B illustrates core insert removal from the tissue flap. FIG. 8C The tubular tissue flap based on the airway scaffold after removal of the core insert

FIGS. 9A-C illustrate the evaluation of tissue formation surrounding an exemplary airway scaffold. FIGS. 9A-B illustrates H&E and MT staining results, respectively, of longitudinal cross-section of the regenerated tubular tissue flap based on the airway scaffold at 6 weeks after implantation (Scale bar, 2 mm; white arrows indicate blood vessels). FIG. 9C is a callout box of a portion of FIG. 9B.

FIGS. 10A-B provide schematics of the expandable open ring shown in FIGS. 4C-D, in which FIG. 10A provides an exploded view and FIG. 10B provides a top view of a portion of the ring, in accordance with some embodiments of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

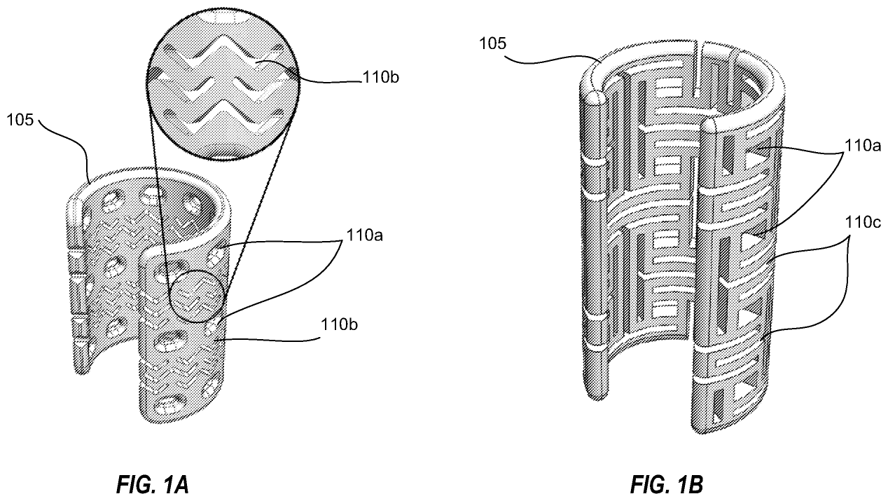

Disclosed herein are implantable devices configured to be implanted into the passageway of a user/patient, including, but not limited to, air passageways and blood passageways. Some of the implantable devices disclosed herein have an open tubular shape. As used herein, the term “open tubular shape” refers to a partially cylindrical shape having an external wall, a first end, and a second end, wherein an opening exist along at least a portion of the external wall from the first end to the second end. Exemplary implantable devices having such an open tubular shape are shown in FIGS. 1-5.

As shown in FIGS. 1A-B & 2A-C, some embodiments of the present disclosure provide an implantable device comprising an external wall 105 having an open tubular shape and a plurality of apertures 110a, 110b, 110c in the external wall 105. The implantable device can be configured to be implanted in a passageway of a user.

The implantable device can be made of many different materials, including, but not limited to biodegradable polymers, non-biodegradable polymers, polycaprolactone (PCL), poly (lactic acid) (PLA), Poly(glycolic acid) PGA, poly (lactic-co-glycolic acid) (PLGA), polyurethane (PU), poly(lactide-co-caprolactone) (PLCL), Polyethylene Glycol (PEG), silicon, polydimethylsiloxane (PDMS), Polyether ether ketone (PEEK), combinations thereof, and the like. In some embodiments, the implantable device can be manufactured via a 3D printing process.

The plurality of apertures 110a, 110b, 110c can have many different shapes to achieve many different functions. For example, in some embodiments, at least a portion of the plurality of apertures can be configured as suture holes 110a for attaching the implantable device to the tissue of the user.

In some embodiments, as shown in FIG. 1A, 2B, & 2C, at least a portion of the plurality of apertures can be stent patterned 110b. The apertures can have many different stent patterns. The present disclosure is not limited to the specific stent patterns shown in the attached figures. The stent patterned apertures 110b can be configured to increase a longitudinal bending flexibility of the implantable device. In some embodiment, the number of stent patterned apertures is correlated with the longitudinal bending flexibility, i.e., a greater number of stent patterned apertures leads to an increase in longitudinal bending flexibility. The stent patterned apertures 110b can also decrease a radial stiffness of the implantable device. In some embodiments, the decrease in radial stiffness can be compensated for by increasing a thickness of the external wall. Due to increased longitudinal bending flexibility, implantable devices with stent patterned apertures can have a relatively longer longitudinal length compared to conventional implantable devices, allowing for use with long passageway defects. The number of the stent-patterns can be determined considering the longitudinal length (length of the passageway defect) and radial rigidity of the device.

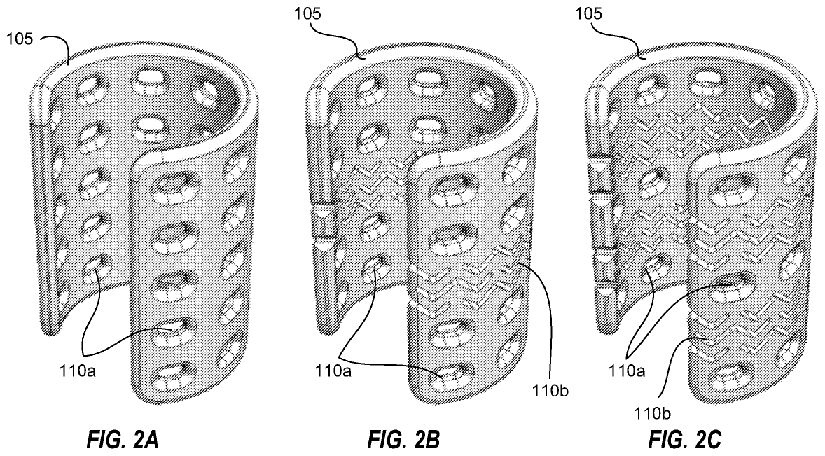

As shown in FIGS. 2B-C, the suture holes 110a can be arranged in rows on the external wall and the stent patterned apertures 110b can be positioned between the rows of suture holes 110a. The configuration of and number of rows can vary in accordance with various embodiments of the present disclosure. For example, in some embodiments, as shown in FIG. 2B, the external wall 105 can have two adjacent rows of suture holes 110a, stent patterned apertures 110b, and two more rows of suture holes 110a. In another embodiments, as shown in FIG. 2C, the external wall can have a single row of suture holes 110a, stent patterned apertures 110b, another row of suture holes 110a, stent patterned apertures 110b, and a final row of suture holes 110a. As discussed above, a greater proportion of stent patterned apertures can increase a longitudinal flexibility of the implantable device.

In some embodiments, as shown in FIG. 1B, a portion of the plurality of apertures in the external wall 105 can have an auxetic pattern 110c. The apertures can have many different auxetic patterns. The present disclosure is not limited to the specific auxetic patterns shown in the attached figures. The auxetic patterned apertures 110c can be configured to provide the implantable device with longitudinal and radial expandability. Implantable devices with auxetic patterned apertures 110c can be particularly useful in applications where the implantable device will remain in the user as the user grows, e.g., trachea, bronchi, esophagus, nerves, and blood vessels of a patient. Additionally, the auxetic patterned apertures 110c can be distributed around a plurality of suture holes 110a on the entire wall 105. The auxetic pattern dimensions including wall thickness, line width, and interval between lines can be adjusted as needed. The auxetic patterns can allow the splinting device to be expanded along a radial direction as the longitudinal length increases, so that it can correspond to the normal growth of the passageway of infants or child patients after external implantation around the passageway defects.

In some embodiments, the stent patterned apertures 110b or the auxetic patterned apertures 110c can also serve as suture holes for attaching the implantable device to the tissue of the user.

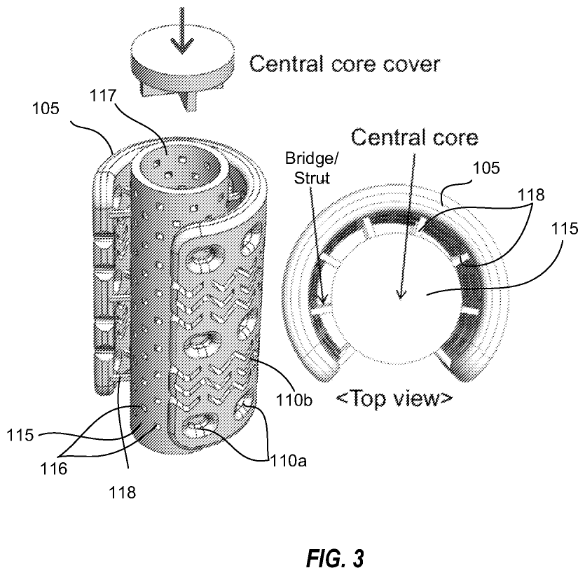

In some embodiments, as shown in FIG. 3, the implantable device can further comprise a central core 115 disposed in an interior volume defined by the external wall 105. Then central core 115 can be employed with embodiments in which the external wall 105 has suture holes 110a, stent patterned apertures 110b, auxetic patterned apertures 110c, or any combination thereof. The central core 115 can be connected to the internal surface of the external wall 105 via one or more struts 118 extending radially outwardly from the central core 115. In some embodiments, the central core 115 can be detachably coupled from the implantable device, such that the implantable device with the coupled central core 115 can be inserted into the user and the central core 115 can be later detached from the external wall 105.

In some embodiments, the central core 115 can comprise a reservoir 117 configured to hold a biological substance to be delivered to the user. For example, the central core 115 can comprise one or more apertures 116 configured to allow the biological substance to exit the reservoir 117 and be delivered to the user.

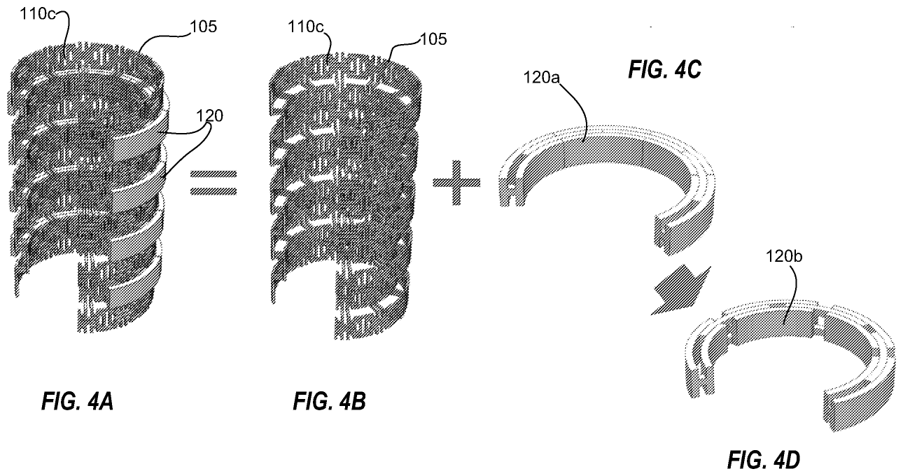

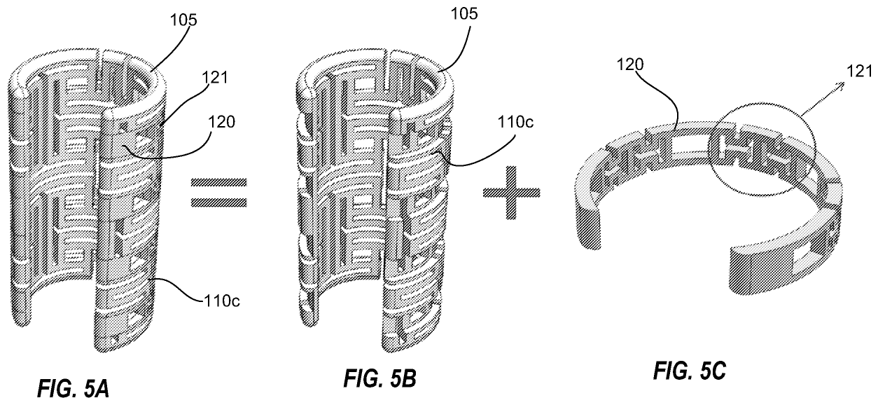

In some embodiments, the implantable device can further comprise an expandable open ring 120, as shown in FIGS. 4A-C, 5A-C, & 10A-B. The expandable open ring can have many different designs that allow it to expand in the radial direction. The present disclosure is not limited to the specific ring design shown in the attached figures. The expandable open ring can be coupled to the external wall 105. For example, in some embodiments, as shown in FIG. 5A, the expandable ring can be integral with the external wall 105. As shown in FIGS. 4C-D, the expandable open ring 120 can be configured to expand radially from a contracted position 120a to an expanded position 120b. In some embodiments, the expandable open ring 120 can be formed from a nonbiodegradable polymer while the external wall can be formed from a biodegradable polymer. Additionally, as shown in FIG. 5C, the expandable open ring 120 can comprise one or more apertures arranged in a zero-Poisson's ratio pattern 121, which can allow for radial expansion.

### Examples

The following examples further illustrate aspects of the present disclosure. However, they are in no way a limitation of the teachings or disclosure of the present disclosure as set forth herein.

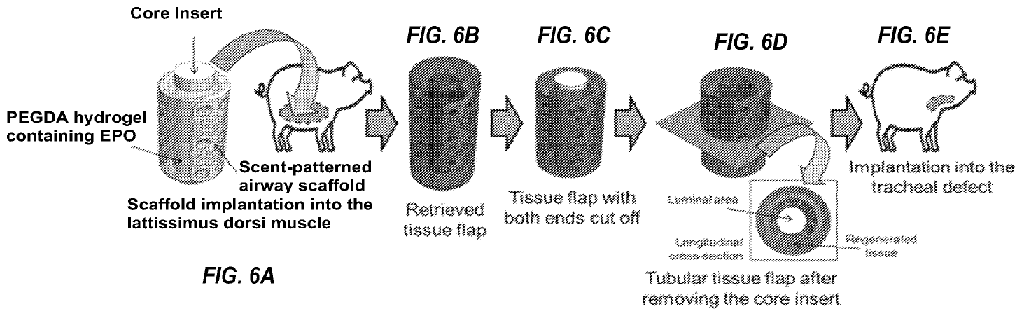

Since the first personalized external airway support device (ASD) was developed based on 3D bioprinting, it has been successfully applied to the patients for treatment of life threatening tracheobronchomalacia (TBM) over the last decade. Ad disclosed below, a rational tissue engineering strategy was developed based on an advanced ASD for long segment tracheal reconstruction. As a framework of the pre-vascularized tubular tissue flap, an airway scaffold having sufficient radial rigidity as well as longitudinal bending flexibility was designed based on the previous ASD and created by selective laser sintering (SLS) based 3D bioprinting. The airway scaffold including a core insert in the luminal area was filled with Poly (ethylene glycol) diacrylate (PEGDA) hydrogel containing 0.24 mg/mL of erythropoietin (EPO) to enhance vascularization and implanted into the latissimus dorsi muscle in a minipig model for a preliminary test (FIGS. 6A-E). After 6 weeks of implantation, we assessed the feasibility of the pre-vascularized tubular tissue flap strategy at a proof-of-concept level.

### Materials And Methods

Materials: Poly-ϵ-caprolactone (PCL, Polyscience Inc., USA) with a mixture of 4% (w/w) hydroxyapatite (HA, Plasma Biotal Ltd., UK) was used for SLS based 3D printing of the airway scaffold with an inner cylinder. PEG, acryloyl chloride (AcCl), Triethylamine (TEA), dithiothreitol (DTT), ammonium persulfate (APS), and N,N,N′,N′-tetramethylethylenediamine (TEMED) were purchased from Sigma Aldrich Inc. (St. Louis, MO, USA). EPO was purchased from Creative Biomart (Shirley, NY, USA).

Airway Scaffold Design and Mechanical Behavior Analysis: The airway scaffold as a framework of the pre-vascularized tubular tissue flap was designed based on the previous implantable ASD. Stent-patterned airway scaffolds were designed with 2.3 mm wall thickness while normal airway scaffold without stent-pattern has 2.0 mm wall thickness (FIG. 2A-C). All airway scaffolds have 32 mm longitudinal length, 15 mm inner diameter, and 90° opening angle. The core insert was additionally added to the luminal area of the airway scaffold to restrict excessive tissue infiltration into the luminal area for a tubular tissue flap creation. The core inserts of a 11 mm diameter were connected to the scaffold wall by a number of bridges having a square cross-section of 500×500 μm² (FIG. 3).

FEA was performed to analyze the effect of stent-pattern on the mechanical properties and behavior of the airway scaffold. Radical compression and three-point bending simulations were performed with two different directions (parallel and perpendicular to the scaffold opening) using FEBio studio version 1.6.0 (Febio.org). 4-node tetrahedral elements were used in the model of the airway scaffolds, and the base PCL material of airway scaffolds was considered linear isotropic elastic with a Young's modulus of 0.116 GPa and a Poisson's ratio of 0.3 in simulation.

Bioprinting of the Airway Scaffolds: The stent-patterned airway scaffolds with and without the core insert were created by a selective laser sintering (SLS) based 3D bioprinting system, Formiga P110 (Electro-Optical Systems (EOS) GmbH, Krailing, Germany). STL files exported from SolidWorks® were imported into Magics software and processed by duplications, translations, rotations, and nesting into labeled sinter boxes on the platform. PSW software (Version 3.6, EOS GmbH) was used to slice the processed STL files into the 100 μm thickness layers. The sliced data was then transferred to the Formiga P110 and the airway scaffolds were created through a laser sintering process using 4 W laser with a scanning speed of 1,500˜2,000 mm/sec.

Mechanical Test: A 5944 Single Column mechanical testing system (Instron Corp., Norwood, MA, USA) with a 2 kN load cell was used for compression tests using the stent-patterned airway scaffolds. Compression at 5 mm/min was applied along the parallel and perpendicular directions to the scaffold opening and load-displacement responses were recorded during the tests.

Hydrogel Preparation: PEGDA was prepared as previously described. PEG (3.4 kD) was dissolved in dichloromethane (DCM, VWR, US) and reacted with AcCl in an 8:1 AcCl to PEG molar ratio. TEA was added dropwise to catalyze the reaction in a 1:1 TEA to AcCl molar ratio to yield linear PEGDA. 15 wt. %/v PEGDA and 20 mol. % DTT, relative to PEGDA, were mixed with phosphate-buffered saline (PBS, ThermoFisher, USA). The solution was incubated at 37° C. for 30 minutes to allow for Michael-Type addition of the DTT with PEGDA. 2 mL of a 0.5 mg/mL solution of EPO was added to the hydrogel solution. PBS was added to achieve a total volume of 4.2 mL. 0.022M ammonium persulfate (APS) and 0.022M TEMED were added to initiate crosslinking. All hydrogel components were sterilized using sterile filters before mixing.

Implantation: PEGDA containing EPO was filled into the gap between the airway scaffold and core insert. Immediately after mixing all hydrogel components, precursor solutions were injected into the custom mold containing the airway scaffold inside. Crosslinking occurred for 20 minutes at room temperature.

Histological Analysis: A Yucatan minipig was sacrificed at 6 weeks after implantation, and the implants were retrieved for histological analysis. Samples were fixed with 10% neutral buffered formalin (NBF) and processed with a TP1020 tissue processor (Leica Biosystems, Wetzlar, Germany) to prepare tissue-embedded paraffin blocks. Blocks were cut into 8 μm thick sections and the tissue sections were stained with hematoxylin & eosin (H&E) and Masson's trichrome (MT). Stained tissue sections were scanned using an Olympus Nanozoomer whole slide scanner (Hamamatsu Photonics, Hamamatsu, Japan) and the scanned images were analyzed using QuPath software.

### Results

### Mechanical Behavior of Stent-Patterned Airway Splints

FEA results showed that the stent-patterned scaffolds with 2.3 mm wall thickness have almost equal or higher radial stiffness compared to the normal airway scaffold with no pattern (FIG. 2A-B). Despite the thicker wall thickness, the stent-patterned scaffolds showed higher bending flexibility than the normal airway scaffold without stent-pattern (FIG. 2C & 3). The airway scaffold with 2 stent-patterns was chosen for further in vivo study as it has almost equal radial stiffness to that of the normal airway scaffold while exhibiting the highest bending flexibility.

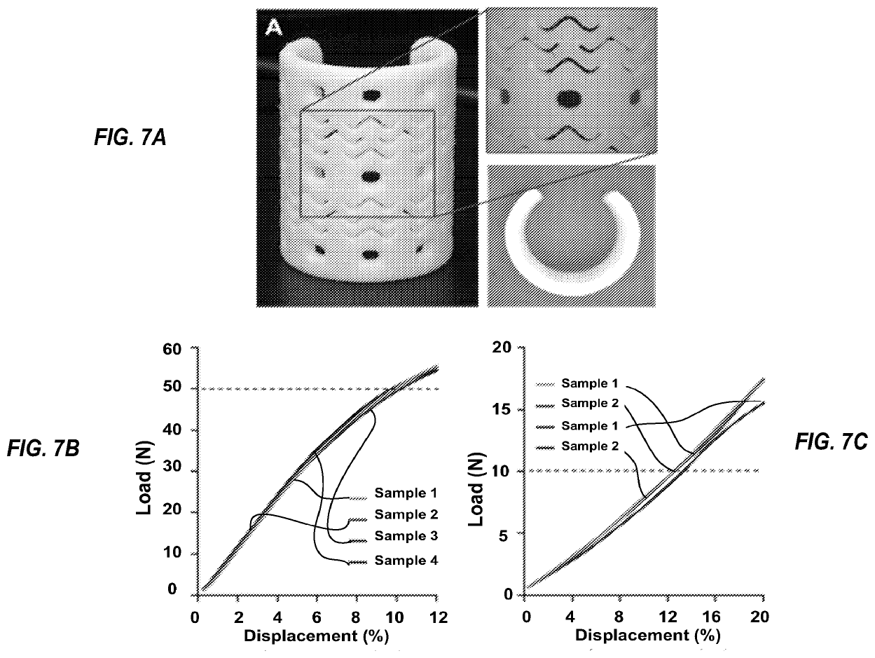

3D Printed Airway Scaffold with 2 Stent-Patterns: The airway scaffold with two stent-patterns was successfully fabricated by SLS based 3D bioprinting (FIG. 7A). The suture holes as well as stent-patterns in the wall were clearly visible. The parallel and perpendicular compression testing results demonstrated that the printed airway scaffold with stent-patterns met mechanical design inputs of allowing less than 40˜50% of gap between the platens under 40˜50 N in parallel compression and 20% of opening angle gap under 10 N in perpendicular compression, respectively (FIG. 7B-C).

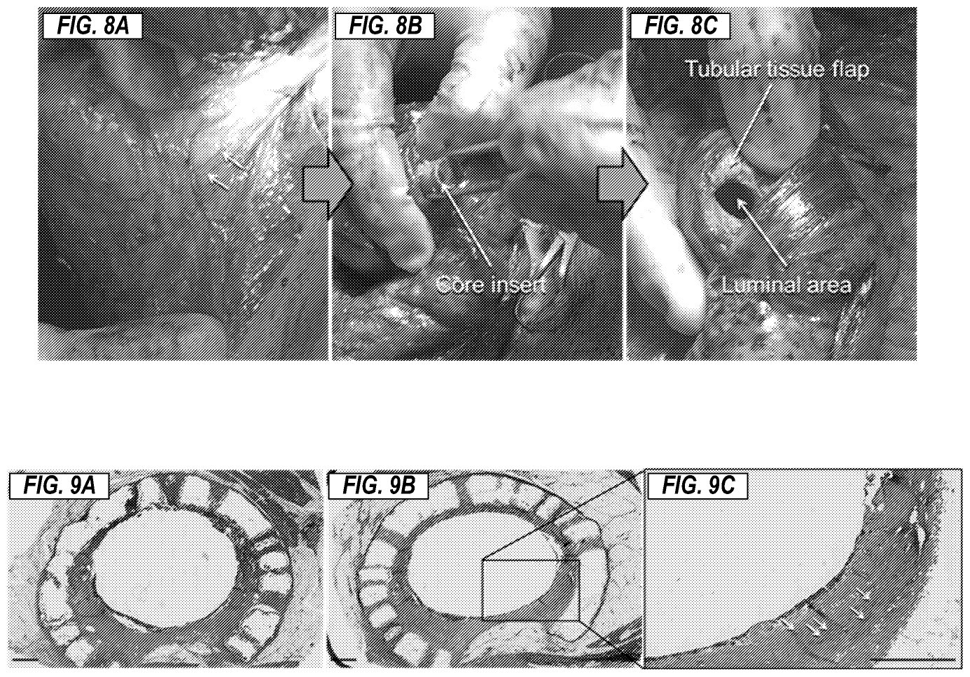

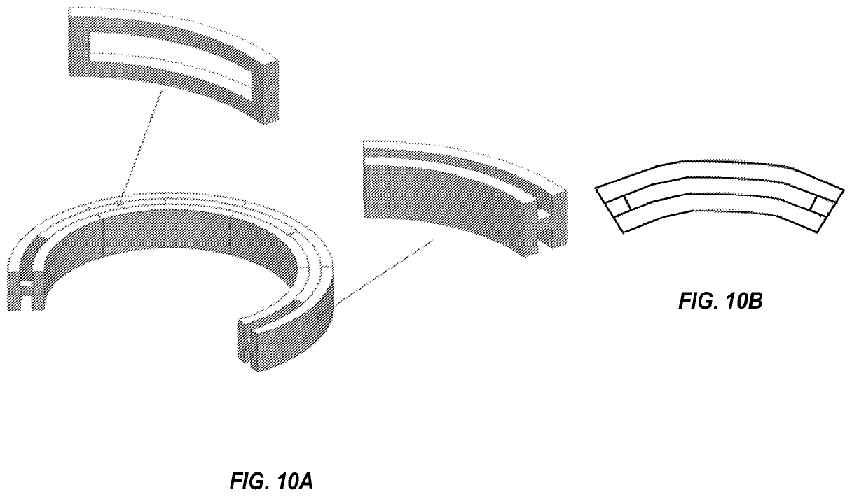

Vascularlized Tubular Tissue Flap: At 6 weeks after implantation, the tissue flap containing the airway scaffold was retrieved. The developed blood vessels were observed on the reconstructed tissue surrounding the airway scaffold exposed after skin resection (FIG. 8A). The core insert was successfully removed from the tissue flap during the retrieval, and the vascularized tubular tissue flap based on the airway scaffold was created (FIG. 8B-C).

H&E image of longitudinal cross-section of the tubular tissue flap indicated that the luminal surface of the airway scaffold was completely covered by reconstructed tissue and the airway scaffold was incorporated with reconstructed surrounding tissues in 6 weeks (FIG. 9A). Muscle tissue formation with muscle fibers and collagen was also confirmed by MT staining result (FIG. 9B). Infiltrated microvessels were also found in the regenerated muscle tissue around the airway scaffold (FIG. 9C).

### Discussion

The airway scaffold discussed above, has an advanced design based on the previous ASD. The ASD can have the form of the open tubular structure, with a plurality of suture holes on the wall, which is designed to support airway defects externally without considering bending flexibility of the native trachea. The current airway scaffold also can have the same form of the open tubular structure as ASD; however, it can have stent-patterns on the wall, which give the airway scaffold an additional bending flexibility. Even though these stent-patterns weaken radial rigidity of the scaffold, it was easily addressed with increased wall thickness. In case of the application in the same manner as the previous ASD, the airway scaffold can address longer stenotic segments than previous ASD as the longitudinal bending flexibility of the airway scaffold assures mechanical stability with a patency of long segmental lesion after external implanting around the long segmental defects.

As the other application, we assessed the potential of the airway scaffold as a framework of the pre-vascularized tubular tissue flap for reconstruction of the tracheal defects after long segmental resection. The stent-patterned airway scaffold with core insert was implanted into a muscle bed and successfully created a vascularized autogenous tubular tissue flap within 6 weeks. EPO in PEGDA hydrogel accelerated the tissue migration from the surrounding muscle tissue and the core insert successfully restricted excessive tissue ingrowth into the luminal area. The core insert was then readily removed from the luminal area of the airway scaffold and the successful formation of the tubular tissue flap with uniform luminal thickness was achieved.

In this study, the stent-pattern application provides the scaffold bending flexibility without changes in patency for an application to the reconstruction of long segment tracheal defect. The number of the stent-patterns applicable to the airway scaffold can depend on the longitudinal length of the airway scaffold which will be determined based on the length of the tracheal defective lesion. Applying a larger number of stent-patterns enhances the bending flexibility of the airway scaffold; however, it simultaneously has an adverse effect on the radial rigidity. The airway scaffold may still have enough radial rigidity to maintain the patency after implantation for the tissue flap creation and further tracheal reconstruction. Therefore, the number of the stent-patterns should be determined considering the longitudinal length (length of the tracheal defect lesion) and radial rigidity of the scaffold

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. In a conventional implantable device comprising:
an external wall having an open tubular shape; and
a plurality of apertures in the external wall, at least a first portion of the plurality of apertures configured as suture holes;
wherein the conventional implantable device is configured to be implanted in a passageway of a human or other animal subject, and upon implantation, has longitudinal bending flexibility, longitudinal expandability, radial rigidity, radial expandability, and patency;
the improvement comprising an improved implantable device comprising the external wall and suture holes, and further comprising:
at least a second portion of the plurality of apertures configured differently from the first portion;
wherein the improved implantable device, upon implantation, has one or more of:
a greater amount of longitudinal bending flexibility than that of the conventional implantable device;
a greater amount of longitudinal expandability than that of the conventional implantable device;
a weaker radial rigidity than that of the conventional implantable device;
a greater amount of radial expandability than that of the conventional implantable device; or
substantially the same patency as the conventional implantable device.

2. The improved implantable device of claim 1, wherein the second portion of the plurality of apertures are configured as stent-patterned apertures; and
wherein the improved implantable device, upon implantation, has:
a greater amount of longitudinal bending flexibility than that of the conventional implantable device;
a weaker radial rigidity than that of the conventional implantable device; and
substantially the same patency as the conventional implantable device.

3.-4. (canceled)

5. The improved implantable device of claim 2, wherein the suture holes are arranged in one or more rows; and
wherein the stent-patterned apertures are arranged in one or more rows different from the one or more rows of suture holes.

6. The improved implantable device of claim 2 further comprising:
at least a third portion of the plurality of apertures configured as auxetic-patterned apertures.

7. The improved implantable device of claim 6, wherein the improved implantable device, upon implantation, has:
a greater amount of longitudinal expandability than that of the conventional implantable device; and
a greater amount of radial expandability than that of the conventional implantable device.

8. The improved implantable device of claim 1, wherein the second portion of the plurality of apertures are configured as auxetic-patterned apertures; and
wherein the improved implantable device, upon implantation, has:
a greater amount of longitudinal expandability than that of the conventional implantable device; and
a greater amount of radial expandability than that of the conventional implantable device.

9.-10. (canceled)

11. In a conventional implantable device comprising:
an external wall having an open tubular shape; and
a plurality of apertures in the external wall, at least a first portion of the plurality of apertures configured as suture holes;
wherein the conventional implantable device is configured to be implanted in a passageway of a human or other animal subject, and upon implantation, has longitudinal bending flexibility, longitudinal expandability, radial rigidity, radial expandability, and patency;
the improvement comprising an improved implantable device comprising the external wall and suture holes, and further comprising:
at least a second portion of the plurality of apertures configured differently from the first portion; and
at least one of:
an expandable open ring coupled to the external wall, the expandable open ring configured to expand radially from a contracted position to an expanded position; or
a central core disposed in an interior volume defined by the external wall;
wherein the improved implantable device, upon implantation, has one or more of:
a greater amount of longitudinal bending flexibility than that of the conventional implantable device;
a greater amount of longitudinal expandability than that of the conventional implantable device;
a weaker radial rigidity than that of the conventional implantable device;
a greater amount of radial expandability than that of the conventional implantable device; or
substantially the same patency as the conventional implantable device.

12. The improved implantable device of claim 11, wherein the expandable open ring is formed from a nonbiodegradable polymer.

13. The improved implantable device of claim 12, wherein the external wall of the implantable device is formed from a biodegradable polymer.

14. The improved implantable device of claim 11, wherein the expandable open ring is integral with the external wall.

15. The improved implantable device of claim 11, wherein the expandable open ring comprises one or more apertures arranged in a zero-Poisson's ratio pattern.

16. (canceled)

17. The improved implantable device of claim 11 further comprising:
one or more struts extending outwardly from the central core and coupled to an inner surface of the external wall.

18. The improved implantable device of claim 17, wherein the central core is detachably coupled to the external wall.

19. The improved implantable device of claim 11, wherein the central core comprises a reservoir configured to hold a biological substance.

20. The improved implantable device of claim 19, wherein the central core comprises one or more apertures configured to allow the biological substance to exit the reservoir.

21. The improved implantable device of claim 1, wherein the improved implantable device is 3D printed.

22. An implantable device for implantation in a passageway of a human or other animal subject comprising:
an external wall defining an interior lumen, the external wall having an open tubular shape;
a plurality of wall apertures in the external wall;
an expandable open ring comprising one or more ring apertures arranged in a zero-Poisson's ratio pattern;
a central core comprising:
a reservoir configured to hold a biological substance; and
one or more core apertures configured to allow the biological substance to exit the reservoir; and
one or more struts extending outwardly from the central core and coupled to an inner surface of the external wall;
wherein:
the expandable open ring is coupled to the external wall;
the expandable open ring is configured to expand radially from a contracted position to an expanded position; and
the central core is disposed in an interior volume defined by the external wall.

23. The implantable device of claim 22, wherein at least a portion of the plurality of wall apertures are configured as one or more of:
suture holes for securing the implantable device in the passageway;
stent-patterned apertures configured to:
increase a longitudinal bending flexibility of the implantable device; and
decrease a radial stiffness of the implantable device; or
auxetic-patterned apertures configured to configured to provide the implantable device with longitudinal and radial expandability.

24.-39. (canceled)

40. The implantable device of claim 22, wherein:
the central core is detachably coupled to the external wall;
the expandable open ring is integral with the external wall;
the expandable open ring is formed from a nonbiodegradable polymer; and
the external wall of the implantable device is formed from a biodegradable polymer.

41.-43. (canceled)

44. The implantable device of claim 22, wherein:
a first portion of the plurality of wall apertures are selected from the group consisting of suture holes, stent-patterned apertures, and auxetic-patterned apertures;
a second portion of the plurality of wall apertures are selected from the group consisting of suture holes, stent-patterned apertures, and auxetic-patterned apertures;
the first portion of the plurality of wall apertures are different than the second portion of the plurality of wall apertures;
the suture holes are configured for securing the implantable device in the passageway;
the stent-patterned apertures are configured to:
increase a longitudinal bending flexibility of the implantable device; and
decrease a radial stiffness of the implantable device; and
the auxetic-patterned apertures are configured to configured to provide the implantable device with longitudinal and radial expandability.
